(19) United States
(12) Patent Application Publication
(10) Pub. No.: US 2024/0306988 A1
(43) Pub. Date: Sep. 19, 2024

(54) Artificial Intelligence Assisted Control of Hemodynamics and Anesthesia in Surgery Patients

(71) Applicant: The Regents of the University of California, Oakland, CA (US)

(72) Inventors: Chih-Ming Ho, Los Angeles, CA (US); Soban Umar, Los Angeles, CA (US); Jure Marijic, Los Angeles, CA (US); Michael Zargari, Woodland Hills, CA (US); Daniel Garcia, Los Angeles, CA (US); Jinyoung Brian Jeong, Los Angeles, CA (US)

(73) Assignee: The Regents of the University of California, Oakland, CA (US)

(21) Appl. No.: 18/571,597

(22) PCT Filed: Jun. 24, 2022
(86) PCT No.: PCT/US2022/073158

§ 371 Date: Dec. 18, 2023

Related U.S. Application Data
(60) Provisional application No. 63/214,476, filed on Jun. 24, 2021.

(51) Int. Cl. A61B 5/00; A61B 5/021; A61K 31/137; A61K 31/21; A61K 31/4468; G16H 20/17; G16H 40/67

(52) U.S. Cl. CPC A61B 5/4848; A61B 5/021; A61B 5/4839; A61B 5/7264; A61K 31/137; A61K 31/21; A61K 31/4468; G16H 20/17; G16H 40/67; A61B 2505/03

(57) ABSTRACT

Systems and methods for artificial intelligence enable control of hemodynamics in an individual are provided. A number of embodiments use a phenotypic response surface (PRS) that describes a physiological response to a vasopressor and/or a vasodilator to guide administration of the vasopressor and/or vasodilator. Additional embodiments determine an individualized response to but not limited to a vasopressor and/or vasodilator based on the change in pressure to a dose based on a population-based average. Some embodiments continually update the PRS and/or individualized sensitivity based on changes in physiological response to the vasopressor and/or vasodilator.

### CROSS REFERENCE TO RELATED APPLICATIONS

This application claims priority to U.S. Provisional Application Ser. No. 63/214,476, entitled “Artificial Intelligence Assisted Control of Hemodynamics and Anesthesia in Surgery Patients,” filed Jun. 24, 2021, which is hereby incorporated by reference in its entirety.

### FIELD OF THE INVENTION

The present invention generally relates to systems and methods for artificial intelligence enabled control of hemodynamics and anesthesia in surgery patients.

### BACKGROUND OF THE INVENTION

Precise management of the hemodynamics of patients undergoing surgery and in the Intensive Care Unit (ICU) is challenging due to a myriad of patient-specific factors. Medications are typically selected to manage hemodynamics based on the medication pharmacokinetics and the dosing is intermittently modified to obtain a desired therapeutic effect. The response of patients to different medications, such as vasopressors and vasodilators, that are used to elevate and reduce blood pressure respectively, is complicated by patients' unique and dynamic physiologic responses to medications that can evolve throughout the course of their surgery or hospitalization.

Among all high-risk surgery patients, around 900,000 patients undergo cardiac surgery per year in the US with post-operative acute kidney injury (AKI) occurring in 5% to 30% of them. Even short durations of an intraoperative Mean Arterial Pressure (MAP) less than 55 mmHg are associated with stroke, acute kidney injury and myocardial injury. In-hospital mortality of patients with AKI is around 20 to 25%. Poor perioperative hemodynamic management is a contributing factor to a number of conditions including post-operative AKI, myocardial infarction, and stroke, which is associated with increased mortality. As such, a significant need exists for effective personalized and targeted hemodynamic management of patients undergoing surgery in the operating room (OR).

### BRIEF SUMMARY OF THE INVENTION

Many embodiments are directed to systems and methods for artificial intelligence enabled control of hemodynamics and anesthesia in surgery patients and applications thereof.

In some aspects, the techniques described herein relate to a method for artificial intelligence enabled control of hemodynamics, including providing a first dose of a blood pressure regulator to an individual, where the first dose is based on a population averaged sensitivity, determining an individualized sensitivity to the blood pressure regulator based on the individual's response to the first dose, and providing a second dose of the blood pressure regulator to the individual, where the second dose is based on the individualized sensitivity.

In some aspects, the techniques described herein relate to a method, where the blood pressure regulator is selected from a vasopressor and a vasodilator.

In some aspects, the techniques described herein relate to a method, where the first dose is a fraction of a full dose of the blood pressure regulator, where a full dose is a population averaged amount of the blood pressure regulator to return the blood pressure to a target blood pressure.

In some aspects, the techniques described herein relate to a method, where the fraction is at least 1/20 of the full dose.

In some aspects, the techniques described herein relate to a method, where the fraction is selected from 1/20, 1/15, 1/10, 1/9, ⅛, 1/7, ⅙, ⅕, ¼, ⅓, and ½ of the full dose.

In some aspects, the techniques described herein relate to a method, where the vasopressor is selected from the group consisting of phenylephrine and norepinephrine and the vasodilator is selected from the group consisting of nitroglycerin and fentanyl.

In some aspects, the techniques described herein relate to a method, where the first dose is provided when a blood pressure of the individual deviates from a target pressure.

In some aspects, the techniques described herein relate to a method, where the second dose returns the blood pressure to a target blood pressure range.

In some aspects, the techniques described herein relate to a method, further including providing a third dose of the blood pressure regulator to the individual upon a deviation from a target pressure, where the third dose is based on the individualized sensitivity.

In some aspects, the techniques described herein relate to a method, further including providing a first dose of a second blood pressure regulator to an individual, where the first dose of the second blood pressure regulator is based on a population averaged sensitivity, determining an individualized sensitivity to the second blood pressure regulator based on the individual's response to the first dose of the second blood pressure regulator, and providing a second dose of the second blood pressure regulator to the individual, where the second dose is based on the individualized sensitivity to the second blood pressure regulator.

In some aspects, the techniques described herein relate to a method, where the blood pressure regulator is a vasopressor and the second blood pressure regulator is a vasodilator.

In some aspects, the techniques described herein relate to a method, where the vasopressor is selected from phenylephrine and norepinephrine and the vasodilator is selected from nitroglycerin and fentanyl.

In some aspects, the techniques described herein relate to a method, where the blood pressure regulator is a vasodilator and the second blood pressure regulator is a vasopressor.

In some aspects, the techniques described herein relate to a method, where the vasopressor is selected from phenylephrine and norepinephrine and the vasodilator is selected from nitroglycerin and fentanyl.

In some aspects, the techniques described herein relate to a method, further including constructing a phenotypic response surface (PRS) describing the individual's response to the blood pressure regulator, and controlling the blood pressure of the individual by providing at least one additional dose of the blood pressure regulator to the individual based on the physiological response described in the PRS.

In some aspects, the techniques described herein relate to a method, further including updating the PRS based on additional physiological response to the blood pressure regulator.

In some aspects, the techniques described herein relate to a method, where the individual is undergoing surgery and on anesthesia.

In some aspects, the techniques described herein relate to a method, where the individual being treated for a serious condition in an Intensive Care Unit or emergency room.

In some aspects, the techniques described herein relate to a method, further including updating the individualized sensitivity to the blood pressure regulator.

In some aspects, the techniques described herein relate to an artificial intelligence enabled system for hemodynamic control, including a physiological monitor configured to measure at least one blood pressure component of an individual, at least one pump configured to administer a blood pressure regulator to the individual, and a computing device in communication with the physiological monitor and the pump operating an artificial intelligence enabled phenotypic response surface (AI-PRS) platform and calculates an individualized sensitivity of the individual to the blood pressure regulator, where the AI-PRS platform constructs a phenotypic response surface (PRS) describing the physiologic response of the individual in reaction to the at least one of the blood pressure regulator, and where the computing device is configured to administer a dose of the blood pressure regulator via the at least one pump upon a change in the at least one blood pressure component measured by the physiological monitor.

In some aspects, the techniques described herein relate to a system, where the at least one blood pressure component is selected from mean arterial pressure and left ventricular systolic pressure.

In some aspects, the techniques described herein relate to a system, where the computing device is configured to maintain a target range of the at least one blood pressure component.

In some aspects, the techniques described herein relate to a system, where the at least one blood pressure component is mean arterial pressure and the target range is 70±10 mmHg.

In some aspects, the techniques described herein relate to a system, where the computing device updates the PRS based on continually monitoring physiological responses to the administration of the blood pressure regulator.

In some aspects, the techniques described herein relate to a system, where the computing device updates the individualized sensitivity based on continually monitoring physiological responses to the administration of the blood pressure regulator.

In some aspects, the techniques described herein relate to a system, where the at least one pump is at least two pumps, where a first pump is configured to administer a vasopressor and a second pump is configured to administer a vasodilator.

In some aspects, the techniques described herein relate to a system, where the blood pressure regulator is a vasopressor selected from phenylephrine and norepinephrine.

In some aspects, the techniques described herein relate to a system, where the blood pressure regulator is a vasodilator selected from nitroglycerin and fentanyl.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIGS. 1A-1B illustrate phenotypic response surfaces (PRSs) of drug responses in accordance with various embodiments.

FIG. 1C provides an AI-PRS function in accordance with various embodiments.

FIGS. 2A-2B illustrate mean arterial blood pressure (MAP) as a function of time with boluses of medication indicated with a best-fit line of the MAP curve shown following medication administration in accordance with various embodiments.

FIG. 2C illustrates left ventricular systolic pressure (LVSP), used as a surrogate for MAP, under control of an artificial intelligence-enabled system in accordance with various embodiments.

FIG. 3A illustrates an exemplary control of mean arterial pressure (MAP) over time in accordance with various embodiments.

FIGS. 3B-3C illustrate an exemplary method for determining an individualized drug sensitivity in accordance with various embodiments.

FIG. 4 illustrates a system for artificial intelligence-enabled control of drug administration in accordance with various embodiments.

FIG. 5 illustrates a method for artificial intelligence-enabled control of drug administration in accordance with various embodiments.

### DETAILED DESCRIPTION OF THE INVENTION

Turning now to the drawings, systems and methods for artificial intelligence enabled control of hemodynamics and anesthesia in surgery patients are described. Many embodiments are directed to the use of artificial intelligence (AI) as a personalized medicine tool to tailor the anesthetic and hemodynamic management of individual patients based on their unique physiology and biochemistry response profiles. The goal in such management typically is to maintain the blood pressure (BP) of a patient within a specified range while the patient is under anesthesia. Typically two different medications can be administered-one to raise the BP and one to lower the BP. The dosage that is provided is calculated based on the specific patient's sensitivity (i.e., response to the particular medication), which can be difficult to determine. In addition, some medications can interact, leading to greater or lesser effect of one or more medications and/or create adverse effects.

Various embodiments are utilized to analyze the hemodynamic data of patients undergoing surgery and use continuously learning AI platform to provide a patient's individual hemodynamic response to medications and to guide choice of medication as well as dose to achieve hemodynamic goals. Here, “inputs” can include amounts or dosages of medications, such, but not limited to, epinephrine, norepinephrine, vasopressin, phenylephrine, nitroglycerin, and/or fentanyl, while mean arterial blood pressure (MAP) is the primary phenotypic “output”.

In several embodiments, an algorithmic approach can be utilized to determine proper dosage level(s) for a medication for a particular patient during surgery. More precise control can be exercised by dosing in two phases. A first dose can be administered based upon a population average sensitivity. A population averaged sensitivity can be calculated based on a variety of factors, such as, but not limited to, previous experimental/empirical data measuring response over a set of previous patients (e.g., how much of a medication used and the resulting blood pressure or change in blood pressure). For example, the population averaged sensitivity can be calculated as the average response to a drug dose for a statistical sample of a population. A population can be defined in a number of ways, including (but not limited to) all humans, a specific subpopulation (e.g., race, gender, sex, ethnicity, country of origin, etc.). In various embodiments, the statistical sample includes at least 50 people, 100 people, 150 people, 250 people, 500 people, 1000 people, or any other number of people to provide a statistically significant measure of a population averaged sensitivity.

In a second phase, some embodiments provide a dosage based upon the patient's individualized sensitivity. Notably, a patient's sensitivity can change over time t, becoming less responsive than average, in some cases dropping five to ten fold. Subsequent dosages in the second phase can utilize artificial intelligence techniques to achieve a target blood pressure range by calculation. For example, AI can be used to estimate a trajectory coming out of a target range. In this way, individual sensitivity for a specific patient can be calibrated dynamically at each point in time. In several embodiments, AI techniques such as AI feedback control, e.g., hill climbing techniques, can be used to calibrate sensitivity of the patient to provide further iterations of doses of medication. Neural networks can be utilized to determine drug-dose response surface. In some embodiments, divide pressure by dose that was given in the first phase to calculate starting point for individualized sensitivity.

### AI-Based Phenotypic Response Surface

Various embodiments utilize an AI-based phenotypic response surface (AI-PRS) platform to prospectively determine a patient's optimal drug and dose combination. (See e.g., U.S. Pat. Pub. No. 2014/0309974 and PCT Pub. No. WO 2021/092057; the disclosures of which are hereby incorporated by reference herein in their entireties.) The clinically validated AI-PRS platform of many embodiments optimizes medication dosing independent of underlying disease pathology through an inherent incorporation of a patient's unique pharmacokinetics and physiologic responses.

Embodiments of this disclosure are directed to identifying optimized combinations of input parameters for a complex system. The goal of optimization of some embodiments of this disclosure can be any one or any combination of reducing labor, reducing cost, reducing risk, increasing reliability, increasing efficacies, reducing side effects, reducing toxicities, and/or alleviating drug resistance, among others. In some embodiments, a specific example of treating diseases of a biological system with optimized drug combinations (or combinatorial drugs) and respective dosages is used to illustrate certain aspects of this disclosure. A biological system can include, for example, an individual cell, a collection of cells such as a cell culture or a cell line, an organ, a tissue, or a multi-cellular organism such as an animal, an individual human patient, or a group of patients. A biological system can also include, for example, a multi-tissue system such as the nervous system, immune system, or cardio-vascular system.

More generally, embodiments of this disclosure can optimize wide varieties of other complex systems by applying pharmaceutical, chemical, nutritional, physical, or other types of stimulations. Applications of embodiments of this disclosure include, for example, optimization of drug combinations, vaccine or vaccine combinations, chemical synthesis, combinatorial chemistry, drug screening, treatment therapy, cosmetics, fragrances, and tissue engineering, as well as other scenarios where a group of optimized input parameters is of interest.

Stimulations can be applied to direct a complex system toward a desired state, such as applying drugs to treat a patient having a disease. The types and the values (e.g., amplitudes or dosages) of applying these stimulations are part of the input parameters that can affect the efficiency in bringing the system toward the desired state. However, N types of different drugs with M dosages for each drug will result in MN possible drug-dosage combinations. To identify an optimized or even near optimized combination by multiple tests on all possible combinations is prohibitive in practice. For example, it is not possible to perform all possible drug-dosage combinations in animal and clinical tests for finding an effective drug-dosage combination as the number of drugs and doses increase.

Embodiments of this disclosure provide a technique that allows a rapid search for optimized combinations of input parameters to guide multi-dimensional (or multi-variate) medical problems, as well as controlling other complex systems with multiple input parameters toward their desired states. An optimization technique can be used to identify at least a subset, or all, optimized combinations or sub-combinations of input parameters that produce desired states of a complex system. Taking the case of combinational drugs, for example, a combination of N drugs can be evaluated to rapidly identify optimized dosages of the N drugs, where N is greater than 1, such as 2 or more, 3 or more, 4 or more, 5 or more, 6 or more, 7 or more, 8 or more, 9 or more, or 10 or more, and so on.

For instance, in liver transplant patients receiving tacrolimus for post-transplant immunosuppression therapy, the utilization of AI-PRS, in accordance with some embodiments, can result in significant reductions in the variation of tacrolimus trough levels compared to patients who received their tacrolimus doses based on a physician-guided regimen. (See e.g., A. Zarrinpar, Sci. Transl. Med. 8, 333ra49 (2016); the disclosure of which is incorporated by reference herein in its entirety.) The AI-PRS platform has also proved successful in the management of patients with prostate cancer through optimizing chemotherapy dosing, such as combinations of ZEN-3694 and enzalutamide, to minimize prostate specific antigen (PSA) levels. (See e.g., A. J. Pantuck, DOI: 10.1002/adtp.201800104, Advanced Therapeutics, (2018), the disclosure of which is incorporated by reference herein in its entirety.)

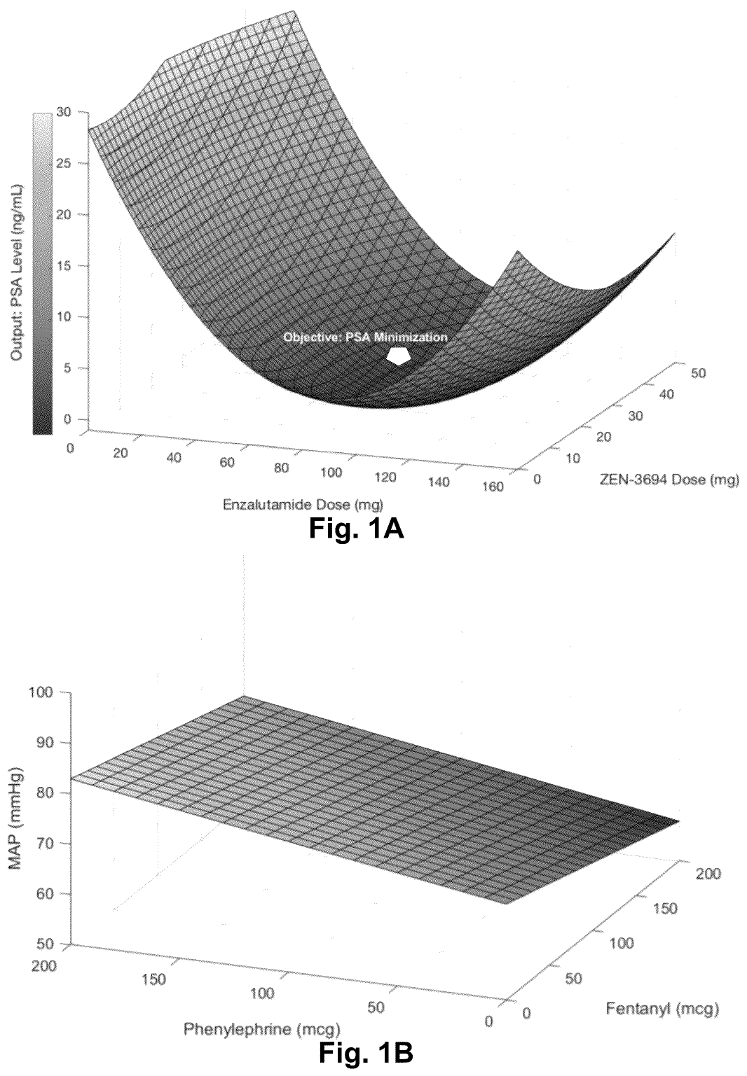

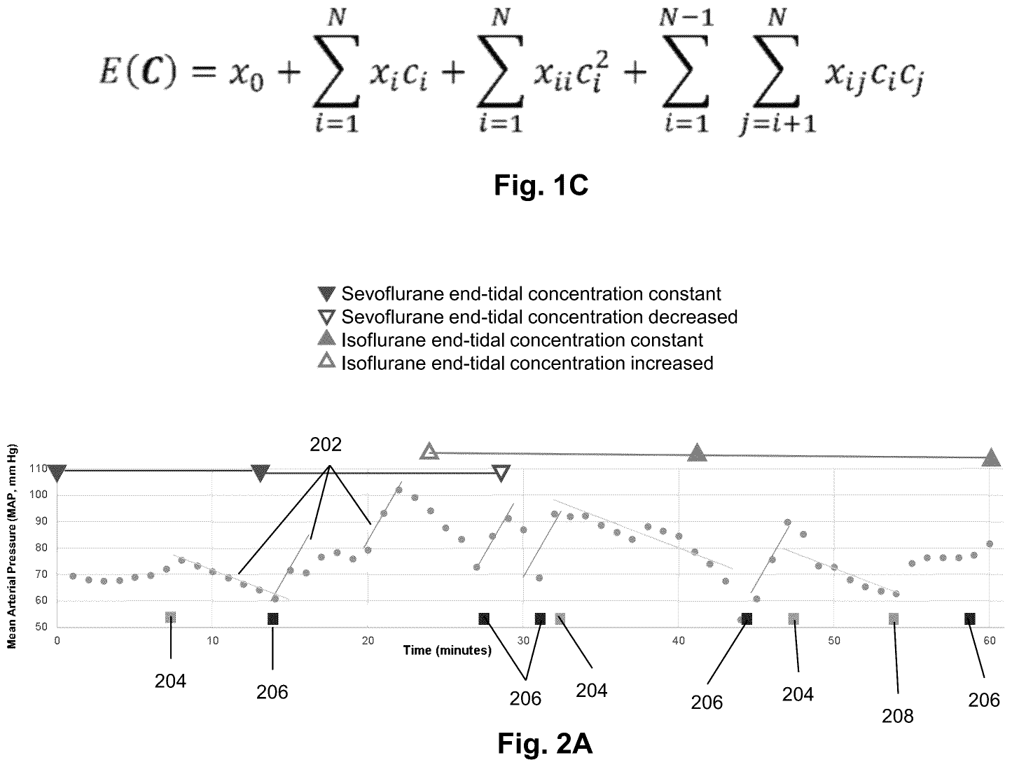

AI-PRS, as used in many embodiments, produces a graph of a three-dimensional smooth surface, referred to as the phenotypic response surface (PRS), and represents a patient's unique physiologic response to therapeutic agents, including but not limited to, blood pressure regulators (e.g., vasopressors and vasodilators), immunosuppressants, chemotherapeutics (including, but not limited to, ZEN-3694 and enzalutamide), anesthesia, and combinations thereof. For example, FIG. 1A illustrates an exemplary embodiment of a PRS profile of a patient with prostate cancer showing the appropriate doses of enzalutamide and ZEN-3694 to minimize the PSA level. Similarly, FIG. 1B illustrates an exemplary embodiment of a PRS profile depicting mean arterial blood pressure (MAP), a blood pressure component, response to phenylephrine and fentanyl administration. In many embodiments, the AI-PRS platform of many embodiments optimizes drug dosing to maximize treatment efficacy over physician-guided treatments. In many embodiments, the PRS is governed by the AI-PRS equation, as illustrated in FIG. 1C, where E(C) is MAP (Mean Arterial Pressure), Cᵢ is Dose of a vasopressor (e.g., phenylephrine) or vasodilator (e.g., nitroglycerin), or medication with effect of lowering blood pressure (e.g., fentanyl), and xₒ, xᵢ, xᵢᵢ are experimental coefficients. Many embodiments incorporate a continuously learning AI platform to provide an individualized drug response and to guide choice of medication and dose to achieve control of one or more specific parameters.

Given the challenges of managing the hemodynamics of patients undergoing surgery or treatment in the ICU, embodiments utilize AI-PRS as a tool for medication selection and dose optimization. Many embodiments present AI-PRS as a personalized therapy and data-driven tool to tailor the anesthetic and hemodynamic management of patients in the operating room and ICU based on their unique physiology and biochemistry response profiles.

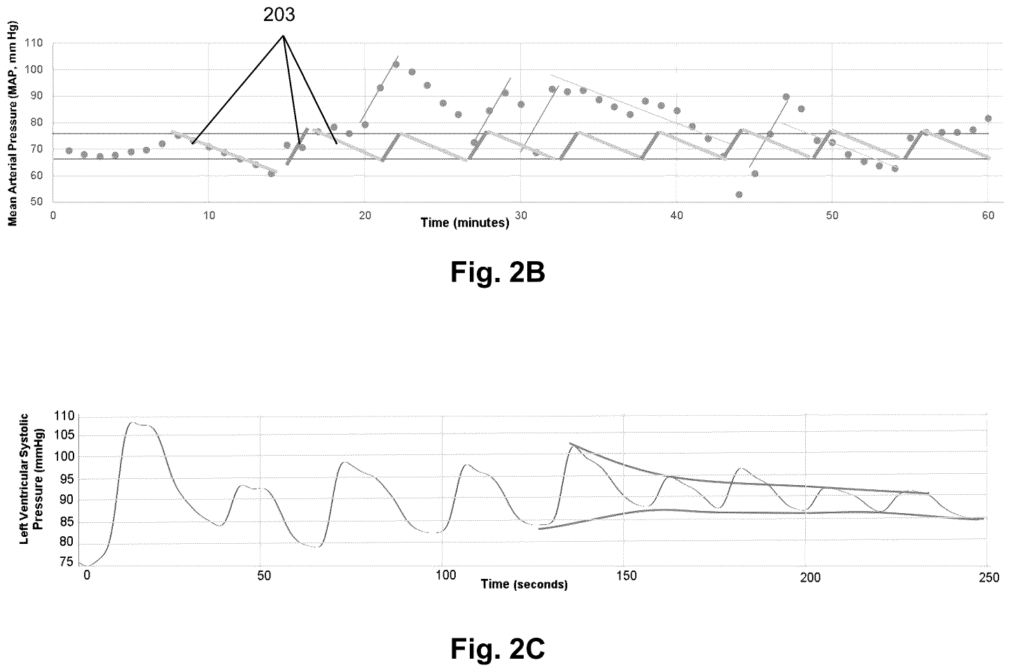

Turning to FIGS. 2A-2C, an exemplary embodiment of a hemodynamic control using AI-PRS is illustrated. In particular, FIG. 2A illustrates MAP plotted against time for a patient undergoing surgery. Trendlines 202 illustrate increasing and decreasing trends of the MAP over the time. Additionally, the timing of sevoflurane and isoflurane anesthetics are plotted in addition to the time of administration of boluses of fentanyl 204, phenylephrine 206, and norepinephrine infusion 208. Based on patient response to drug boluses, FIG. 2B illustrates an idealized level of control of MAP with trendlines 203 showing MAP control within a target range of approximately 65 mmHg and approximately 75 mmHg. However, the target range for blood pressure maintenance can be modified based on a patient. For example, some embodiments may maintain a mean target pressure of between 60-80±10 mmHg. Additionally, the target range may vary depending on the specific blood pressure component being measured (e.g., MAP versus LVSP). Additionally, FIG. 2C illustrates exemplary results of left ventricular systolic pressure (LVSP) control of an embodiment, where the blood pressure has decreased fluctuation under control of the embodiment.

### Personalized Control of Hemodynamics

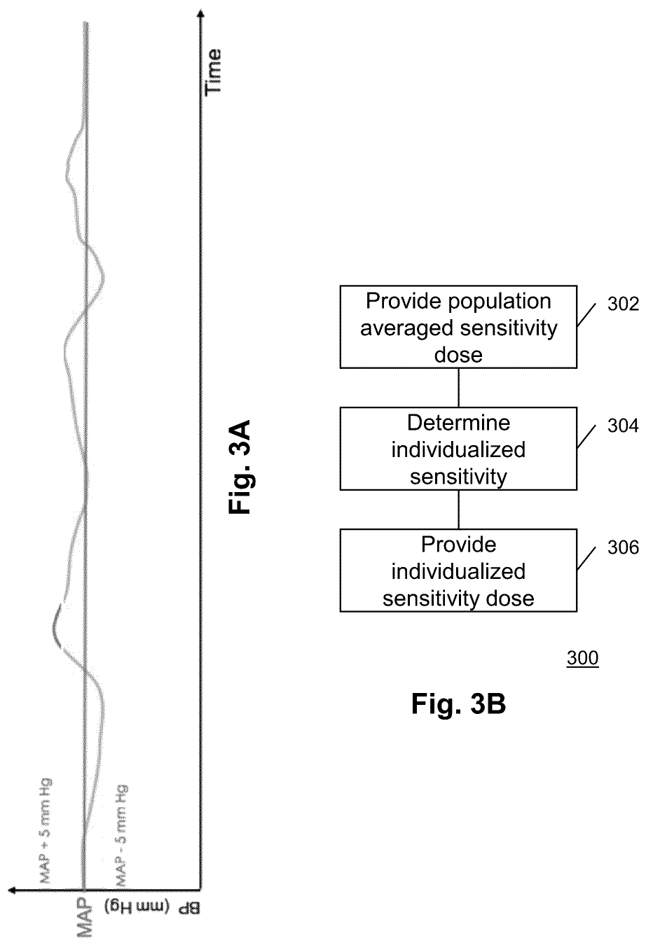

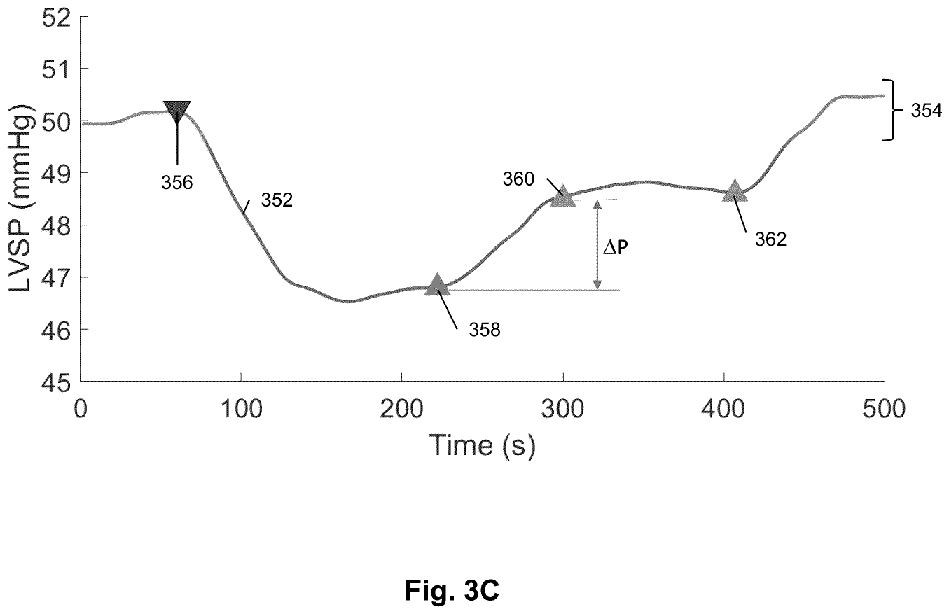

While AI-PRS technologies and platforms can bring BP to a desired MAP in prospective animal test, there is still a large diversity among patients regarding drug sensitivity. This diversity can cause each patient to react very differently to one or more drugs as compared to a population average. As such, many embodiments provide a personalized control of hemodynamics (PCH) for precise control of BP for surgery patients, especially high-risk patients. Because of this diversity, many embodiments tailor the anesthetic management of patients based on their unique physiology and biochemistry response profiles. Embodiments implementing PCH enable guidance of hemodynamics (e.g., blood pressure) within a narrow and appropriate range personalized to each patient, as illustrated in FIG. 3A, which provides an exemplary MAP of an individual over the course of surgery.

FIG. 3B illustrates an exemplary method 300 for providing PCH to an individual. In method 300, an individual is under anesthesia and/or under hemodynamic control. In many embodiments, when a deviation from a target blood pressure is identified (or induced), the process provides a dose of a blood pressure regulator (e.g., vasodilator or vasopressor) based on a population averaged sensitivity (302). In many such embodiments, the population averaged sensitivity is based on an AI-PRS platform or other method to determine a populational average. Certain individuals may have a higher sensitivity to a drug, such that a full dose will cause a deviation in the opposite direction (e.g., a full dose will cause an overshoot of the target BP). As there is a risk for overcorrection or overshooting the target BP, many embodiments provide less than a target dose to correct BP to the target BP. In some such embodiments, the dose is 1/20, 1/15, 1/10, 1/9, ⅛, 1/7, ⅙, ⅕, ¼, ⅓, or ½ of the full dose based on population averaged sensitivity to return the blood pressure to a target blood pressure.

Upon providing a dose based on population averaged sensitivity 302, the individual is monitored to identify a specific response in that individual. In certain individuals the response is smaller than the population averaged sensitivity, larger than the population averaged sensitivity, or equal to the population averaged sensitivity. At 304, many embodiments determine an individualized sensitivity based on the response to the dose based on a population averaged sensitivity. In numerous embodiments, the individualized sensitivity is equal to the change in pressure based on the dose provided at 302. A sample equation is provided as:

$$S = Δ P / D$$

where, S is sensitivity, ΔP is the difference in pressure, and D is the dose provided at 302.

Once an individualized sensitivity is determined, certain embodiments provide a dose based on the individualized sensitivity at 306.

It should be noted that the method illustrated in FIG. 3B is merely exemplary and that one of skill in the art would understand that various embodiments may include additional features, duplicate certain features, omit certain features, perform certain features multiple times, and/or perform certain features simultaneously (or nearly simultaneously). For example, some embodiments update (e.g., redetermine) an individualized sensitivity later on in the surgery or control, such as if a person becomes more (or less) sensitive to one or more drugs being provided (e.g., a vasopressor and/or a vasodilator). In some such embodiments, the individualized sensitivity is updated (or redetermined) after every dose of a drug is provided to the individual.

An exemplary graphical representation of an experimental determination of individualized sensitivity is illustrated in FIG. 3C. In FIG. 3C, blood pressure, as measured by left ventricular systolic pressure (LVSP) is plotted 352 graphed over time, and a target blood pressure range 354 is illustrated as a range of approximately 50-51 mmHg-LVSP, in this exemplary instance, is used as a surrogate for systolic blood pressure As shown in FIG. 3C, a cardiac event is induced at 356. In this exemplary graph, a drop in left ventricular systolic pressure (LVSP) is induced at 356. In doing so, the LVSP falls below the target range 354. At 358, a dose of a vasopressor based on the population averaged sensitivity is provided, which causes an increase of LVSP 352. At 360, the LVSP 352 plateaus, allowing for a determination of an individualized sensitivity, such as described herein (e.g., S=ΔP/D). Based on the determination of an individualized sensitivity, a dose of a vasopressor based on the individualized sensitivity is provided at 362. The individualized dose returns the LVSP 352 to the target range 354.

While FIG. 3C provides an example, where the blood pressure drops below of target range 354, and a vasopressor is used to regain a target pressure, a similar process can be used to determine individualized sensitivity to vasodilators or other drugs to reduce blood pressure, when blood pressure deviates above a target range 354.

### Systems for Hemodynamic Control

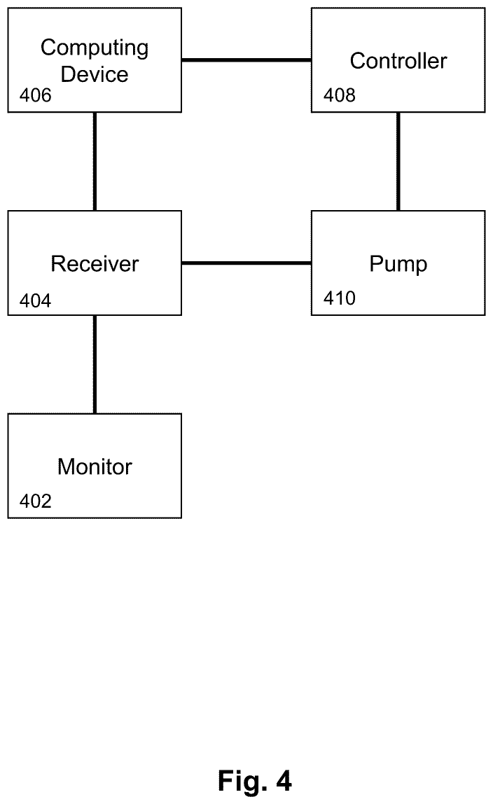

Turning to FIG. 4, various embodiments analyze hemodynamic data of patients undergoing surgery and use continuously learning AI platform to provide a patient's individual hemodynamic response to medications and to guide choice of medication as well as dose to achieve hemodynamic goals. Many embodiments possess a physiological monitor 402 to obtain physiological data about an individual under treatment. Certain embodiments monitor blood pressure and/or other clinical parameters. Certain embodiments utilize a monitor selected from GE Solar line, a LiDCo device, an Edwards Lifesciences EV-1000, and/or any other monitor known in the art.

Additional embodiments possess a receiver 404 in communication with and to obtain data from a monitor 402. Further embodiments include a computing device 406 including a processor and memory. A computing device 406 can process physiologic data coming from a receiver 404, such as to determine what medicine to administer (e.g., vasopressor or vasodilator) based on an individual response profile that particular medicine, when to administer the medicine, and what dose to administer. In many embodiments, the computing device can operate the AI-PRS platform to identify physiologic responses to a dose of medicine or drug and/or any interactions between multiple drugs. Such methods of determining dose using an AI-PRS platform in accordance with many embodiments is described elsewhere herein. In further embodiments, computing device 406 can determine an individualized sensitivity to the drugs, and in some embodiments to update the individualized sensitivity, such as described elsewhere herein.

Further embodiments include one or more controllers 408 in communication with a computing device 406. Controllers 408 of many embodiments are configured to select a particular drug, timing, and/or dose as determined by a computing device 406. Further embodiments can administer a medicine or drug via a pump 410, such as a peristaltic pump or any other pump sufficient for administering a particular drug based on timing of dose, rate of administration, and size of dose. In many embodiments, one or more pumps 410 are in communication with a receiver 404, such that the receiver further receives data regarding which medicine, dose amount, timing of dose, rate of administration of a dose, and/or any other information that a pump may have regarding medicine or drug administration. In such embodiments, a computing device 406 can further correlate dosing data with physiologic data received from a physiologic monitor 402.

It is contemplated within some embodiments that certain configurations may be combined into a single device, rather than individualized components, such that certain embodiments are contained as a single, integrated computing device comprising a monitor and pump, such that phycological data from a monitor is communicated directly to a processor, which further controls one or more integrated pumps to administer one or more drugs to a patient.

### Methods for Treating an Individual

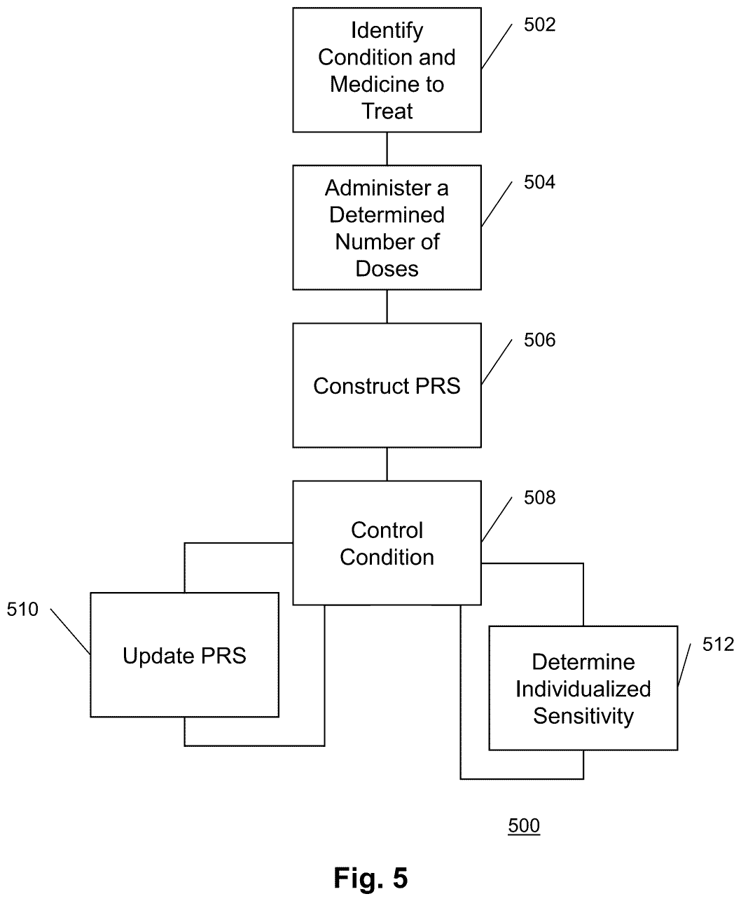

Turning to FIG. 5, some embodiments are directed to methods 500 to treat an individual based on an individual's physiological response to one or more drugs, including interactions between these drugs. At 502 of many embodiments, a treatable condition is identified along with one or more drugs for treatment. In some embodiments, the treatable condition is management of a disease, disorder, or physiological condition, such as a cancer, infections (e.g., viral and/or bacterial), blood pressure, diabetes, psychological/psychiatric disorders, and/or pain. Cancers can include cancers of the bladder, prostate, breast, or any other form of cancer, while infections include viral infections such as HIV, Herpes simplex 1, Herpes simplex 2, coronaviruses, including SARS-CoV-2, and other viruses, while bacterial infections include pneumococcal bacteria, tuberculosis, and other chronic bacterial infections. Additionally, blood pressure management can include during surgical procedures, Intensive Care Unit admission, or whenever a patient becomes hemodynamically unstable where control of a target blood pressure component (e.g., MAP) is useful for better recovery from the surgical procedure or illness. Additionally, certain embodiments can identify pain disorders for treatment or management. Drugs for treatment of the condition include any relevant drug or combination of drugs for treatment, such as anti-inflammatories, antivirals, antibiotics, antipsychotics, vasopressors, vasodilators, blood pressure reducing agent, anesthesia, narcotics, opioids, insulin, steroids, any other drug relevant for treatment, and combinations thereof. In certain embodiments, the condition is controlling blood pressure of an individual during surgery, where the drugs consists of a vasopressor and a blood pressure reducing agent (e.g., vasodilator or drug with effect of reducing blood pressure). In some of these embodiments the vasopressor is selected from the group consisting of phenylephrine, norepinephrine and the blood pressure reducing agent is selected from the group consisting of nitroglycerin and fentanyl. Addition embodiments include administration of blood products, intravenous fluids, pain medication, positive inotrope (to increase myocardial contractility), inovasodilator (to increase myocardial contractility and increase vasodilation), a diuretic (to get rid of excess fluid) any other treatment for a condition being monitored, and combinations thereof.

At 504, many embodiments administer a determined number (or set) of doses to an individual. In some embodiments, the timing, dose size, and which drug to administer is determined via an AI-PRS platform, such as described herein and in U.S. Pat. Pub. No. 2014/0309974 and PCT Pub. No. WO 2021/092057, cited above. An AI-PRS platform of many embodiments produces a PRS curve for an individual, based on physiologic response to the one or more drugs to be administered to an individual. Depending on the number of drugs or medicines to provide, the amount of data points to produce a PRS varies. As noted elsewhere herein, the AI-PRS platform of some embodiments is based on the equation illustrated in FIG. 1C.

At 506, many embodiments construct a PRS based on physiologic response to the one or more drugs. In many embodiments, the PRS is constructed using the AI-PRS platform upon administering the one or more drugs are administered to an individual and measuring the physiological response produced by the one or more drugs. In many embodiments, the PRS identifies the physiological response of the one or more drugs as well as any interactions between drugs being administered that affect the efficacy of one or more of the drugs. Construction of a PRS is described elsewhere herein.

At 508, further embodiments control of a condition identified in 502 with the drug or drugs also identified in 502, based on the PRS constructed in 506. Control in accordance with certain embodiments involves administering one or more of the drugs to produce its respective physiological response in the individual to maintain control of the condition or disease. In some embodiments, control is maintaining a target parameter within a specific range (e.g., MAP of 70±5 mmHg, 70±10 mmHg, etc.), while other embodiments control means maintaining a maximum or minimum parameter such as viral load or PSA.

Many embodiments update the PRS at 510 by continually monitoring physiological responses to the administration of the one or more drugs to the individual, which allow for continually improving control over the condition at 508.

Additional embodiments determine an individualized sensitivity of the patient to one or more of the provided drugs at 512. As noted herein, the individualized sensitivity provides for determination of a more accurate dose of one or more provided drugs (e.g., vasopressor and/or vasodilator for blood pressure management). Such determination can be used to control the condition at 508. With an individualized sensitivity, more accurate dosing can be provided to the individual to maintain tighter control of the condition (e.g., blood pressure). For example, a smaller dose of the drug can be provided at the onset of a deviation (increase or decrease in blood pressure), such that the deviation does not exceed a target range and/or returns to a target pressure without exceeding a target range. Additional embodiments update the individualized sensitivity (e.g., periodically and/or continually) during a procedure (e.g., surgery) to continue to provide tight and accurate control of the condition. Continuous acquisition of the responses to the administration of a medicine in combination with acquisition of physiological data will allow for continuous improvement in predictive power of various embodiments.

In many embodiments, determination of a an individualized sensitivity happens multiple times (e.g., iteratively and/or continually) throughout the control process or procedure. For example, some embodiments update the individualized sensitivity after every dose of a drug, while some embodiments update the individualized sensitivity when the response deviates from the expected response by a certain threshold. The threshold can be a percent difference from the expected response or a scalar measurement away from the expected response. For example, an individualized sensitivity could be updated or redetermined if the expected response differs by 5%, 10%15%, 20%, 25%, 30%, or other percent deviation (e.g., 10% deviation would be ±0.5 mmHg, when the expected response is 5 mmHg). In certain embodiments, the individualized sensitivity when the response deviates from the target blood pressure by a set percent, such as 5%, 10% 15%, 20%, 25%, 30%, or other percent deviation (e.g., if the target blood pressure is 70 mmHg, a 10% deviation would be ±7 mmHg). Certain embodiments elect a direct difference in the response (versus a proportional or percentage deviation in the response) from the drug—for example, if the actual response differs from the target response or target pressure by 0.5 mmHg, 1.0 mmHg, 1.5 mmHg, 2.0 mmHg, 2.5 mmHg, 3.0 mmHg, 3.5 mmHg, 4.0 mmHg, 4.5 mmHg, 5.0 mmHg, or more. Various embodiments provide control over target pressures and ranges by a medical practitioner, as for a particular individual or blood pressure component (e.g., MAP, LVSP, etc.) being measured may necessitate a different target or range.

It should be noted that certain embodiments may combine some features, repeat some features, or omit some features of method 500, as necessary for a particular purpose, such that controlling a condition, where multiple administrations of the one or more drugs may be necessary to maintain control of the condition.

### Doctrine of Equivalents

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

What is claimed is:

1. A method for artificial intelligence enabled control of hemodynamics, comprising:
providing a first dose of a blood pressure regulator to an individual, wherein the first dose is based on a population averaged sensitivity;
determining an individualized sensitivity to the blood pressure regulator based on the individual's response to the first dose; and
providing a second dose of the blood pressure regulator to the individual, wherein the second dose is based on the individualized sensitivity.

2. The method of claim 1, wherein the blood pressure regulator is selected from a vasopressor and a vasodilator.

3. The method of claim 1, wherein the first dose is a fraction of a full dose of the blood pressure regulator, wherein a full dose is a population averaged amount of the blood pressure regulator to return the blood pressure to a target blood pressure.

4. The method of claim 3, wherein the fraction is at least 1/20 of the full dose.

5. The method of claim 3, wherein the fraction is selected from the group consisting of: 1/20, 1/15, 1/10, 1/9, ⅛, 1/7, ⅙, ⅕, ¼, ⅓, and ½ of the full dose.

6. The method of claim 2, wherein the vasopressor is selected from the group consisting of phenylephrine and norepinephrine and wherein the vasodilator is selected from the group consisting of nitroglycerin and fentanyl.

7. The method of claim 1, wherein the first dose is provided when a blood pressure of the individual deviates from a target pressure.

8. The method of claim 7, wherein the second dose returns the blood pressure to a target blood pressure range.

9. The method of claim 1, further comprising providing a third dose of the blood pressure regulator to the individual upon a deviation from a target pressure, wherein the third dose is based on the individualized sensitivity.

10. The method of claim 1, further comprising:
providing a first dose of a second blood pressure regulator to an individual, wherein the first dose of the second blood pressure regulator is based on a population averaged sensitivity;
determining an individualized sensitivity to the second blood pressure regulator based on the individual's response to the first dose of the second blood pressure regulator; and
providing a second dose of the second blood pressure regulator to the individual, wherein the second dose is based on the individualized sensitivity to the second blood pressure regulator.

11. The method of claim 10, wherein the blood pressure regulator is a vasopressor and the second blood pressure regulator is a vasodilator.

12. The method of claim 11, wherein the vasopressor is selected from the group consisting of phenylephrine and norepinephrine and wherein the vasodilator is selected from the group consisting of nitroglycerin and fentanyl.

13. The method of claim 10, wherein the blood pressure regulator is a vasodilator and the second blood pressure regulator is a vasopressor.

14. The method of claim 13, wherein the vasopressor is selected from the group consisting of phenylephrine and norepinephrine and wherein the vasodilator is selected from the group consisting of nitroglycerin and fentanyl.

15. The method of claim 1, further comprising:
constructing a phenotypic response surface (PRS) describing the individual's response to the blood pressure regulator; and
controlling the blood pressure of the individual by providing at least one additional dose of the blood pressure regulator to the individual based on the physiological response described in the PRS.

16. The method of claim 15, further comprising updating the PRS based on additional physiological response to the blood pressure regulator.

17. The method of claim 1, wherein the individual is undergoing surgery and on anesthesia.

18. The method of claim 1, wherein the individual being treated for a serious condition in an Intensive Care Unit or emergency room.

19. The method of claim 1, further comprising updating the individualized sensitivity to the blood pressure regulator.

20. An artificial intelligence enabled system for hemodynamic control, comprising:
a physiological monitor configured to measure at least one blood pressure component of an individual;
at least one pump configured to administer a blood pressure regulator to the individual; and
a computing device in communication with the physiological monitor and the pump operating an artificial intelligence enabled phenotypic response surface (AI-PRS) platform and calculates an individualized sensitivity of the individual to the blood pressure regulator;
wherein the AI-PRS platform constructs a phenotypic response surface (PRS) describing the physiologic response of the individual in reaction to the at least one of the blood pressure regulator; and
wherein the computing device is configured to administer a dose of the blood pressure regulator via the at least one pump upon a change in the at least one blood pressure component measured by the physiological monitor.

21. The system of claim 20, wherein the at least one blood pressure component is selected from mean arterial pressure and left ventricular systolic pressure.

22. The system of claim 20, wherein the computing device is configured to maintain a target range of the at least one blood pressure component.

23. The system of claim 22, wherein the at least one blood pressure component is mean arterial pressure and the target range is 70±10 mmHg.

24. The system of claim 20, wherein the computing device updates the PRS based on continually monitoring physiological responses to the administration of the blood pressure regulator.

25. The system of claim 20, wherein the computing device updates the individualized sensitivity based on continually monitoring physiological responses to the administration of the blood pressure regulator.

26. The system of claim 20, wherein the at least one pump is at least two pumps, wherein a first pump is configured to administer a vasopressor and a second pump is configured to administer a vasodilator.

27. The system of claim 20, wherein the blood pressure regulator is a vasopressor selected from the group consisting of phenylephrine and norepinephrine.

28. The system of claim 20, wherein the blood pressure regulator is a vasodilator selected from the group consisting of nitroglycerin and fentanyl.
